(19) United States
(12) Patent Application Publication
(10) Pub. No.: US 2022/0240816 A1
(43) Pub. Date: Aug. 4, 2022

(54) NON-INVASIVE MEASURING DEVICE, METHOD, AND PROGRAM

(71) Applicant: MEDICAL PHOTONICS CO., LTD., Kita-ku, Sapporo-shi, Hokkaido (JP)

(72) Inventors: Koichi SHIMIZU, Fukuoka (JP); Kazuya IINAGA, Hokkaido (JP)

(21) Appl. No.: 17/612,419

(22) PCT Filed: Jun. 2, 2020
(86) PCT No.: PCT/JP2020/021723

§ 371 Date: Nov. 18, 2021

(30) Foreign Application Priority Data
Jun. 4, 2019 (JP) 2019-104467

(51) Int. Cl. A61B 5/1455; G06F 17/11

(52) U.S. Cl. CPC A61B 5/1455; A61B 2560/0223; A61B 2562/0233; G06F 17/11

(57) ABSTRACT

A non-invasive measuring device is provided with: an irradiation unit which is for irradiating a specific position on a test subject with light; a light intensity detection unit which is disposed at a position on a blood vessel of the test subject for detecting a first light intensity of light; a light intensity detection unit which is disposed at a position not located on a blood vessel of the test subject for detecting a second light intensity of light; and a control unit which calculates a third light intensity of the blood in a blood vessel from the first light intensity and the second light intensity, a correct attenuation coefficient from a temporary attenuation coefficient obtained from the third light intensity by means of a calibration function, and an equivalent scattering coefficient and an absorption coefficient separately from the attenuation coefficient through measurement of multiple wavelengths.

### TECHNICAL FIELD

The present invention relates to a noninvasive measuring device, method and program.

### BACKGROUND ART

Non-Patent Literature 1 discloses the principle of measurement of the blood turbidity (reflecting in-blood lipid concentration) from the spatial distribution of reflectance R(ρ) (intensity of backscattered light produced at body surface when light is incident on single point on body surface). The reflectance R(ρ) for a transmitter-receiver interval ρ is given by Numerical Formula 1.

$$R ( ρ ) = \frac{{S}_{0}}{2 {μ}_{s}^{′}} ( {μ}_{eff} + \frac{1}{ρ} ) \frac{1}{{ρ}^{2}} {e}^{- {μ}_{eff} ρ} \qquad [ Numerical Formula 1 ]$$

### CITATION LIST

### Patent Literature

Patent Literature 1: Japanese Patent No. 6,241,853

### Non-Patent Literature

Non-Patent Literature 1: SHIMIZU Koichi, Analysis of Light Propagation in Biological Tissue, Japanese Journal of Optics, Vol. 41, No. 8, pp. 414-423, 2012

### SUMMARY OF INVENTION

### Technical Problem

The approach described above, however, has the following three primary problems, which force the blood turbidity measurement to be of low accuracy. (1) The reflectance measured by the approach described above does not allow measurement of pure blood turbidity because the reflectance contains not only backscattered light from the blood but backscattered light from living body tissue excluding the blood. (2) The principle described in Non-Patent Literature 1, on which the approach described above is based, is derived from diffusion approximation, and the principle works well with living body tissue excluding blood but cannot be applied to blood for which the approximation condition does not hold true. (3) The approach described above provides an attenuation coefficient μ_{eff}, and the value of an absorption coefficient μₐ is required to accurately determine an equivalent scattering coefficient μₛ′, which represents blood turbidity. That is, to accurately measure the blood turbidity, the absorption coefficient μₐ also needs to be measured at the same time.

The present invention has been made to solve the three points (1) to (3) described above, and an object of the present invention is to achieve highly accurate, noninvasive measurement.

### Solution to Problem

A noninvasive measuring device according to the present invention includes a light radiator that radiates light to a predetermined position on a subject, optical intensity detectors that are disposed in positions above a blood vessel of the subject and detect a first optical intensity of light emitted from the subject in positions separate at predetermined intervals from a position irradiated with the light from the light radiator, optical intensity detectors that are disposed in positions not above the blood vessel of the subject and detect a second optical intensity of light emitted from the subject in positions separate at predetermined intervals from the position irradiated with the light from the light radiator, and a control unit that calculates a third optical intensity from blood in the blood vessel by using the first optical intensity and the second optical intensity.

A noninvasive measuring program according to the present invention causes a computer in a device including a light radiator that radiates light to a predetermined position on a subject, optical intensity detectors that are disposed in positions above a blood vessel of the subject and detect a first optical intensity of light emitted from the subject in positions separate at predetermined intervals from a position irradiated with the light from the light radiator, and optical intensity detectors that are disposed in positions not above the blood vessel of the subject and detect a second optical intensity of light emitted from the subject in positions separate at predetermined intervals from the position irradiated with the light from the light radiator to carry out the process of calculating a third optical intensity from blood in the blood vessel by using the first optical intensity and the second optical intensity.

A noninvasive measuring method according to the present invention includes radiating light to a predetermined position on a subject, detecting a first optical intensity of light emitted from the subject in positions on a blood vessel of the subject and separate at predetermined intervals from a position irradiated with the light, detecting a second optical intensity of light emitted from the subject in positions not on the blood vessel of the subject and separate at predetermined intervals from the position irradiated with the light, and calculating a third optical intensity from blood in the blood vessel by using the first optical intensity and the second optical intensity.

### BRIEF DESCRIPTION OF DRAWINGS

FIG. 1 shows the configuration of a noninvasive measuring device according to an embodiment.

FIG. 2 shows the arrangement of an optical intensity detector.

FIG. 3 shows that light is scattered by in-blood lipids.

FIG. 4 is a block diagram of a control unit.

FIG. 5 shows Numerical Formula 5 graphed versus a transmitter-receiver interval ρ.

FIG. 6 is a flowchart of noninvasive measurement according to the embodiment.

### DESCRIPTION OF EMBODIMENT

A noninvasive measuring device, program, and method according to an embodiment will be described below in detail with reference to the drawings.

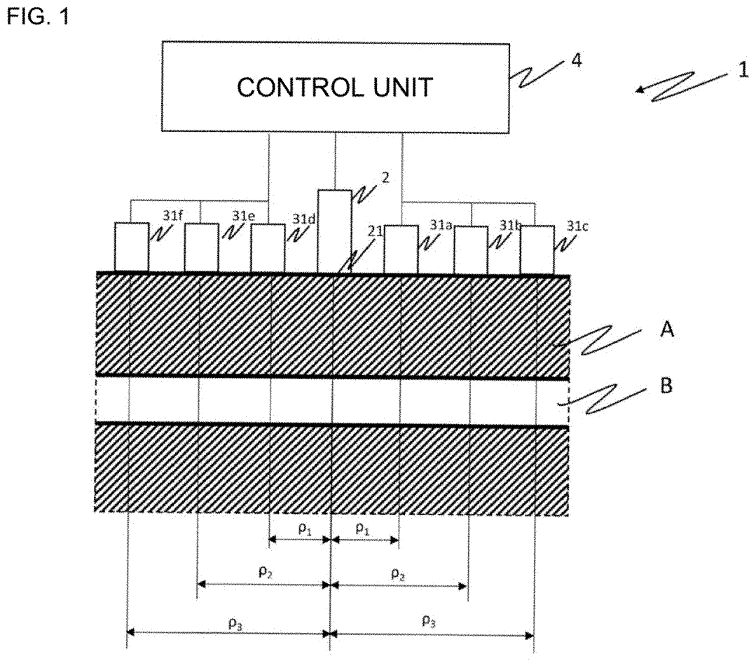

FIG. 1 shows the configuration of the noninvasive measuring device according to the embodiment. FIG. 1 does not show a second optical intensity detector group 32, in which second optical intensity detectors 32a to 32f are aligned in a row.

A noninvasive measuring device 1 includes a light radiator 2, an optical intensity detector 3, and a control unit 4, as shown in FIG. 1. In the drawings, reference character A represents a living body, and reference character B represents a blood vessel in the living body.

The light radiator 2 includes a light source that radiates light to a predetermined radiation position 21 on the predetermined site of the living body from a point outside the living body toward the interior of the living body. The light radiator 2 may be controlled by the control unit 4 in terms of the wavelength and radiation intensity of the light to be radiated.

The light radiator 2 in the embodiment can arbitrarily adjust the length of time, for example, for which the light is radiated continuously or in the form of pulsed light.

The light radiator 2 may be formed of a light source having a fixed wavelength. The light radiator 2 may be the combination of a plurality of light sources that output light fluxes having different wavelengths or may output the mixture of light fluxes having a plurality of wavelengths. The light radiator 2 is, for example, a fluorescent lamp, an LED, a laser, an incandescent lamp, an HID, or a halogen lamp. The illuminance of the light from the light radiator 2 may be controlled by the control unit 4.

The light radiator 2 is an LED (light emitting diode) in the embodiment. The light radiator 2 in the embodiment radiates light having a first wavelength (λ₁) and light having a second wavelength (λ₂), which differs from the first wavelength. The number of light radiator 21 may be one, or a plurality of light radiators 21 may, for example, form a first optical intensity detector group 31 and a second optical intensity detector group 32, which will be described later.

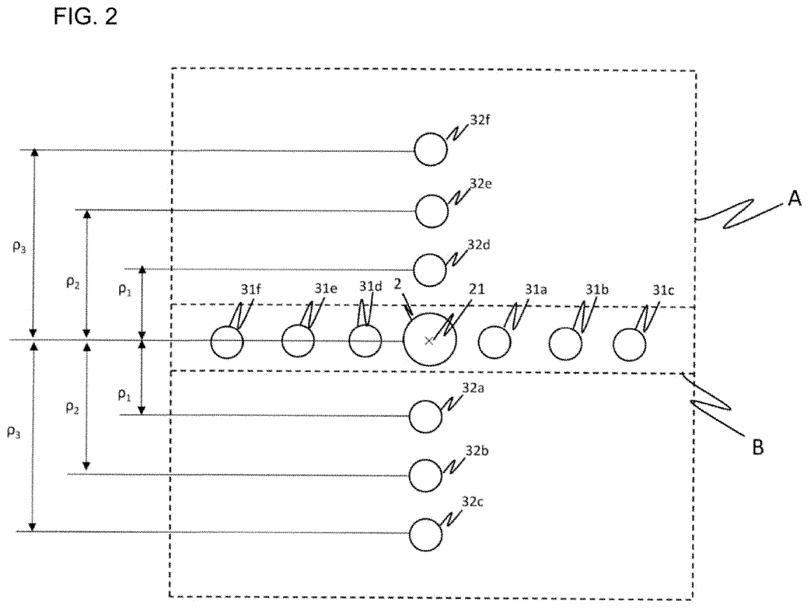

FIG. 2 shows the arrangement of the optical intensity detector 3. The dotted lines in the figures indicate a blood vessel B in a living body A. The optical intensity detector 3 includes detectors arranged in the form of a cross on the surface of the living body (on the skin), as shown in FIG. 2. The optical intensity detector 3 receives light emitted from the living body toward the space outside the living body and detects the optical intensity of the light. The optical intensity detector 3 may be formed of photodiodes, a CCD, or a CMOS device.

The optical intensity detector 3 includes the first optical intensity detector group 31, in which first optical intensity detectors 31a to 31f located at the living body surface above the blood vessel B in the living body A and aligned in a row in the direction along the blood vessel, and the second optical intensity detector group 32, in which second optical intensity detectors 32a to 32f aligned in a row in the direction perpendicular to the first optical intensity detector group 31 (that is, in positions separate away from blood vessel).

The first optical intensity detectors 31a to 31f are disposed at different distances from the center of the radiation position 21, to which the light radiator 2 radiates the light. In the embodiment, the first optical intensity detectors 31a to 31f are arranged in a single plane along a straight line at predetermined intervals from the radiation position 21, as shown in FIG. 1.

The distance from the radiation position 21 to the first optical intensity detectors 31a and 31d is ρ₁, the distance from the radiation position 21 to the first optical intensity detectors 31b and 31e is ρ₂, and the distance from the radiation position 21 to the first optical intensity detectors 31c and 31f is ρ₃.

The second optical intensity detectors 32a to 32f are disposed at different distances from the center of the radiation position 21, to which the light radiator 2 radiates the light. In the embodiment, the second optical intensity detectors 32a to 32f are arranged in a single plane along a straight line at predetermined intervals from the radiation position 21, as shown in FIG. 2.

The distance from the radiation position 21 to the second optical intensity detectors 32a and 32d is ρ₁, the distance from the radiation position 21 to the second optical intensity detectors 32b and 32e is ρ₂, and the distance from the radiation position 21 to the second optical intensity detectors 32c and 32f is ρ₃.

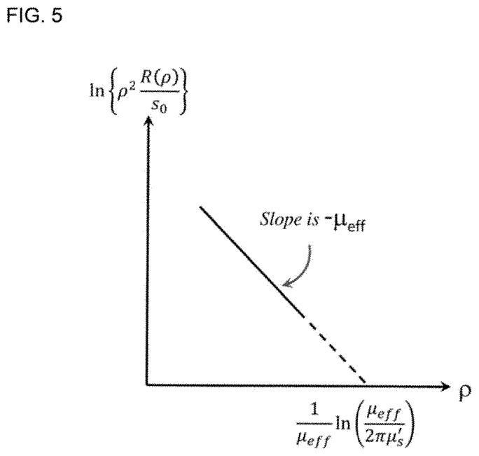

In the embodiment, the number of first optical intensity detectors 31 and the number of second optical intensity detectors are each set at six, but not necessarily. In the embodiment, an attenuation coefficient μ_{eff} is estimated from the gradient of the graph in FIG. 5. Therefore, at least two detectors only need to be present on each side of the light incident point, and the greater the number of detectors, the more accurate the estimation of the attenuation coefficient μ_{eff}.

In related art, in which photodetectors are disposed at the body surface along a vein under observation, the backscattered light scattered through the blood is measured as much as possible, and the measured value is used as the blood reflectance. The actually measured backscattered light, however, contains not only the backscattered light from the blood but not a little amount of backscattered light from the living body tissue around the blood vessel. In view of the fact described above, the reflectance containing as little backscattered light as possible from the vessel is measured around the blood vessel, and the contribution of the measured reflectance to the reflectance is excluded. Pure reflectance from the blood in the vessel can thus be determined.

In the embodiment, the second optical intensity detectors 32a to 32f are disposed in the direction perpendicular to the direction in which the vessel extends, as a method for measuring the “reflectance containing as little backscattered light as possible.” The arrangement of the second optical intensity detectors 32a to 32f is not limited to that described above, and the light source and the second optical intensity detectors 32a to 32f may be disposed along a line shifted from the extending blood vessel in parallel thereto.

The light received by the optical intensity detector 3 is converted into photocurrent, which is processed by the control unit 4.

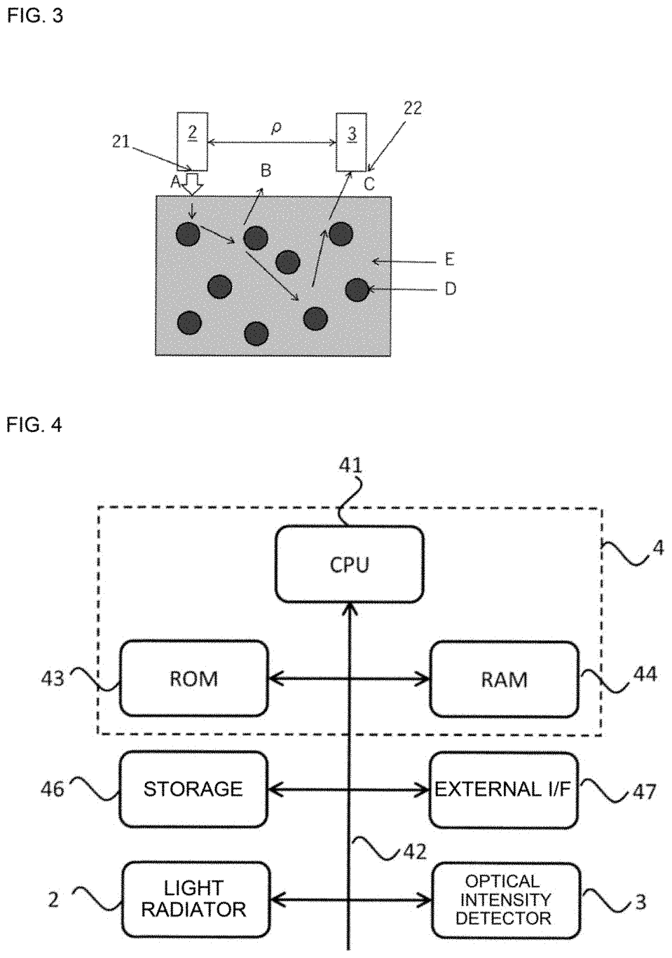

A predetermined distance p is set between the radiation position 21, where the living body is irradiated with the light, and a detection position 31, where the intensity of the light emitted from the blood (E in FIG. 3) in the living body is detected, as shown in FIG. 3. The thus set predetermined distance p suppresses the influence of the light directly emitted from the living body (B in FIG. 3), which is the radiated light (A in FIG. 3) reflected off the surface of the living body and scatterers in the vicinity of the surface. After the radiated light reaches the depth where the blood is present, the light is scattered by the lipids in the blood (D in the figure), and the backscattered light is detected at the surface of the living body.

In the case where a plurality of first optical intensity detectors and a plurality of second optical intensity detectors are provided, the detectors are not necessarily arranged linearly as long as the detectors are disposed at different distances roughly around the radiation position 21, and the arrangement of the detectors can be selected as appropriate from a circular arrangement, a wavy arrangement, a zigzag arrangement, and other arrangements. The first radiation-detection distance ρ₁, the second radiation-detection distance ρ₂, and the third radiation-detection distance ρ₃, which are each the distance from the radiation position 21 to the corresponding one of the first and second optical intensity detectors, the interval between the first optical intensity detectors, the interval between the second optical intensity detectors are not each limited to a fixed distance, and the optical intensity detectors may instead be continuously arranged.

Furthermore, in the embodiment, the first optical intensity detector group 31, in which the first optical intensity detectors 31a to 31f are aligned in a row, and the second optical intensity detector group 32, in which the second optical intensity detectors 32a to 32f are aligned in a row, are perpendicular to each other, but not necessarily, and the first optical intensity detector group 31 and the second optical intensity detector group 32 only needs to be so configured that the first optical intensity detectors 31a to 31f are above the blood vessel B and the second optical intensity detectors 32a to 32f are away from the blood vessel B.

For example, when the first optical intensity detectors 31a to 31f are above the vessel B and the second optical intensity detectors 32a to 32f are away from the vessel B, the first optical intensity detector group 31, in which the first optical intensity detectors 31a to 31f are aligned in a row, and the second optical intensity detector groups 32, in which the second optical intensity detectors 32a to 32f are aligned in a row, can be shifted from each other in parallel to each other.

The configuration of a control system of the noninvasive measuring device 1 will next be described. FIG. 4 is a block diagram of the control unit of the noninvasive measuring device according to the embodiment. A CPU (central processing unit) 41, a ROM (read only memory) 43, a RAM (random access memory) 44, a storage 46, an external I/F (interface) 47, the light radiator 2, and the optical intensity detector 3 are connected to each other via a system bus 42. The CPU 41, the ROM 43, and the RAM 44 form the control unit (controller) 4.

The ROM 43 stores in advance a program executed by the CPU 41 and thresholds used by the CPU 41.

The RAM 44 has an area where the program executed by the CPU 41 is developed, a variety of memory areas, such as a work area where the program processes data, and other areas.

The storage 46 stores data, such as sensed and calculated optical intensities and μ_{eff}. The storage 46 may be an internal memory that stores information in a nonvolatile manner, such as an HDD (hard disk drive), a flash memory, and an SSD (solid-state drive).

The external I/F 47 is an interface for communication with an external device, for example, a client terminal (PC). The external I/F 47 only needs to be an interface that performs data communication with an external device and may, for example, be an instrument (such as USB memory) locally connected to the external device or a network interface for communication via a network.

At the surface of the living body, the first optical intensity detectors 31a to 31f immediately above the blood vessel measure reflectance Rₒₙ(ρ) and transmit the reflectance Rₒₙ(ρ) to the control unit 4. The second optical intensity detectors 32a to 32f, which are separate from the blood vessel, measure reflectance R_{off}(ρ) and send the reflectance R_{off}(ρ) to the control unit 4.

Let α(ρ) and β(ρ) be a contribution factor of blood reflectance R_{blood}(ρ) to Rₒₙ(ρ) and R_{off}(ρ), respectively. The contribution factors α(ρ) and β(p) are determined in advance by simulations or experiments. Furthermore, ratios kₛ and kₐ between the equivalent scattering coefficient and the absorption coefficient at two wavelengths to be used are also determined in advance from values in literatures or from experiments performed by the users.

The control unit 4 uses Rₒₙ(ρ) measured by the first optical intensity detectors 31a to 31f and R_{off}(ρ) measured by the second optical intensity detectors 32a to 32f to determine the blood reflectance R_{blood}(ρ) excluding the contribution of living body tissue. The method for calculating the reflectance R_{blood}(ρ) is as follows.

Numerical formula 2 shows that the reflectance Rₒₙ provided from the first optical intensity detectors 31a to 31f, which are located immediately above the blood vessel and extending therealong, is formed of the blood reflectance R_{blood}, living body tissue reflectance Rₜᵢₛₛᵤₑ, and the proportions α and (1−α) thereof.

Rₒₙ(ρ)=αR_{blood}(ρ)+(1−α)Rₜᵢₛₛᵤₑ(ρ) [Numerical Formula 2]

Numerical formula 3 shows that the reflectance R_{off} provided from the second optical intensity detectors 32a to 32f, which are disposed in the direction perpendicular to the direction in which the first optical intensity detectors 31a to 31f are disposed or are shifted in parallel to the first optical intensity detectors 31a to 31f, is formed of the blood reflectance R_{blood}, the living body tissue reflectance Rₜᵢₛₛᵤₑ, and the proportions β and (1−β) thereof.

R_{off}(ρ)=βR_{blood}(ρ)+(1−β)Rₜᵢₛₛᵤₑ(ρ) [Numerical Formula 3]

The values α and β (both are functions of transmitter-receiver interval ρ) are determined in advance by Monte Carlo simulations or experiments. Numerical Formula 4, which is derived from Numerical Formulae 3 and 4, allows determination of the blood reflectance R_{blood} excluding the contribution of the living body tissue by using the measured Rₒₙ and R_{off}.

$${R}_{blood} ( ρ ) = \frac{( 1 - β ) {R}_{on} - ( 1 - α ) {R}_{off}}{α - β} \qquad [ Numerical Formula 4 ]$$

The control unit 4 estimates the attenuation coefficient μ_{eff} based on the blood reflectance R_{blood}(ρ).

Patent Literature 1 discloses the principle of the measurement of the blood turbidity (reflecting in-blood lipid concentration) from the spatial distribution of the reflectance R(ρ) (intensity of backscattered light produced at body surface when light is incident on single point on body surface). The reflectance R(ρ) for the transmitter-receiver interval ρ is given by Numerical Formula 1 described above.

Therefore, R(ρ) provided for the transmitter-receiver interval ρ is measured and normalized by the amount of incident light S₀, and then the quantity on the left side of Numerical Formula 5 is calculated.

$$ln \{ {ρ}^{2} \frac{R ( ρ )}{{S}_{0}} \} = - {μ}_{eff} ρ + ln ( \frac{{μ}_{eff}}{2 {μ}_{s}^{′}} ) \qquad [ Numerical Formula 5 ]$$

When Numerical Formula 5 is graphed versus the transmitter-receiver interval ρ, the graph is a straight line as shown in FIG. 5, and the gradient of the graph is a negative value −μ_{eff} of the attenuation coefficient μ_{eff}.

The relationship among the attenuation coefficient μ_{eff}, the absorption coefficient μₐ, and the equivalent scattering coefficient μₛ′ is given by Numerical Formula 6 below.

μ_{eff}=\sqrt{3μₐ(μₛ′+μₐ)} [Numerical Formula 6]

Numerical Formula 6, when the absorption coefficient μₐ is known, determines the equivalent scattering coefficient μₛ′ from the attenuation coefficient μ_{eff}. Since the blood turbidity is directly reflected in μₛ′, the blood turbidity can be evaluated by measuring the reflectance R(ρ) and determining the attenuation coefficient μ_{eff}.

The control unit 4 calculates the left side ln{ρ²R_{blood}(ρ)/S₀} of Numerical Formula 5 from the light-transmission-light-reception interval ρ, the amount of incident light S₀, and the blood reflectance R_{blood}(ρ). FIG. 5 shows the graphed Numerical Formula with ln{ρ²R_{blood}(ρ)/S₀} representing the vertical axis and ρ representing the horizontal axis. Since the gradient of the graph in FIG. 5 is the negative value of the attenuation coefficient −μ_{eff}, the attenuation coefficient μ_{eff} can be estimated.

The control unit 4 determines a calibration coefficient k, which eliminates an approximation error of the diffusion approximation, by using the following calibration function. A correct, approximation-error-free attenuation coefficient μ_{eff} is provided by multiplying the attenuation coefficient μ_{eff,meas} provided in the process described above by the calibration coefficient k. That is, μ_{eff}=kμ_{eff,meas}.

A calibration curve function that eliminates an error resulting from the method based on the diffusion approximation is provided as shown by Numerical Formula 7 below.

k=ae^{-bμ}^{_{eff meas}}+c [Numerical formula 7]

When μ_{eff} ranges from 1.00 to 2.00, a, b, and c are given as follows: a=55.7; b=4.63; and c=1.00. Symbol e represents the exponential function.

The correct attenuation coefficient μ_{eff} can thus be determined by first estimating a temporary attenuation coefficient μ_{eff,meas} from the blood reflectance R_{blood} and then multiplying μ_{eff,meas} by the calibration coefficient k provided from the calibration function expressed by Numerical Formula 7.

The control unit 4 carries out the process described above by using light sources that output light having two wavelengths λ₁ and λ₂ to estimate attenuation coefficients μ_{eff}(λ₁) and μ_{eff}(λ₂). The control unit 4 uses the estimated μ_{eff}(λ₁) and μ_{eff}(λ₂) and an equivalent scattering coefficient ratio kₛ and an absorption coefficient ratio kₐ, which are prepared in advance, to determine equivalent scattering coefficients μₛ′(λ₁) and μₛ′(λ₂) and absorption coefficients μₐ(λ₂) and μₐ(λ₂) at the two wavelengths.

The equivalent scattering coefficients μₛ′(λ₁) and μₛ′(λ₂) and the absorption coefficients μₐ (λ₁) and μₐ(λ₂) are separately determined from μ_{eff}(λ₁) and μ_{eff}(λ₂) provided in the process described above by measuring the reflectance at a plurality of wavelengths (two wavelengths λ₁ and λ₂). The value kₛ is the ratio between the equivalent scattering coefficients at the two wavelengths, and the value kₐ is the ratio between the absorption coefficients at the two wavelengths, that is, kₛ=μₛ′(λ₂)/μₛ′(λᵢ), and kₐ=μₛ(λ₂)/μₐ(λ₁). The kₛ and kₐ are determined in advance values in literatures or from experiments.

The control unit 4 separates and calculates μₐ(λ₁) by using Numerical Formula 8.

$${μ}_{a} ( {λ}_{1} ) = \sqrt{\frac{{μ}_{eff} ( {λ}_{2} ) - {k}_{a} {k}_{s} {μ}_{eff} ( {λ}_{1} )}{3 {k}_{a} ( {k}_{a} - {k}_{s} )}} \qquad [ Numerical Formula 8 ]$$

The control unit 4 separates and calculates μₛ′(λ₁) by using Numerical Formula 9.

$${{μ}_{s}}^{′} ( {λ}_{1} ) = \frac{{{μ}_{eff}}^{2} ( {λ}_{1} )}{3 {μ}_{a} ( {λ}_{1} )} - {μ}_{a} ( {λ}_{1} ) \qquad [ Numerical Formula 9 ]$$

The control unit 4 quantifies the degree of optical scattering, such as turbidity of a target object, from the equivalent scattering coefficients and identifies and quantifies the substances that form the target object from the absorption coefficients.

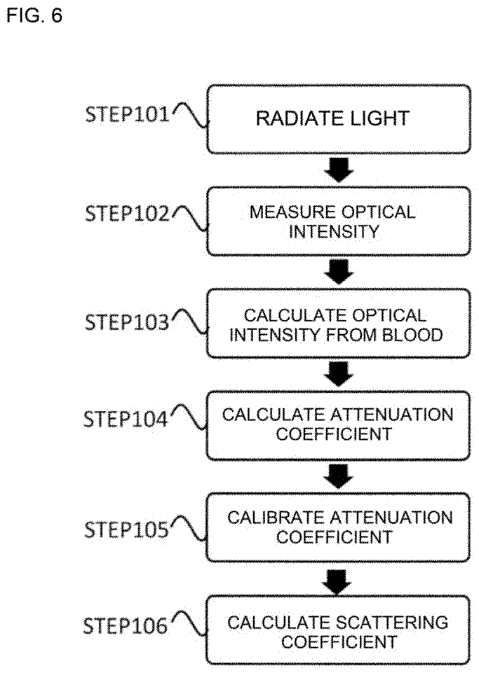

The noninvasive measuring device 1 having the configuration described above performs noninvasive measurement based on a program stored in advance. FIG. 6 is a flowchart of the noninvasive measurement according to the embodiment. The program may be stored in a storage medium.

The light radiator 2 radiates radiation light toward a predetermined site of a living body from a point outside the living body toward the interior of the living body. The light radiator 2 may radiate light having a first wavelength (λ₁) and light having a second wavelength (λ₂), which differs from the first wavelength (step 101).

The first optical intensity detectors 31 measure the optical intensity (reflectance) in positions above a blood vessel of the living body and separate at predetermined intervals from a radiation position irradiated with the radiated light, and the second optical intensity detectors 32 measure the optical intensity (reflectance) in positions that are not above the blood vessel of the living body and separate at predetermined intervals from the radiation position irradiated with the radiated light (step 102).

The control unit 4 determines the blood reflectance R_{blood}(ρ) excluding the contribution of the living body tissue by using Rₒₙ(ρ) measured by the first optical intensity detectors 31 and R_{off}(ρ) measured by the second optical intensity detectors 32 (step 103). The method for calculating the reflectance has been described above.

The control unit 4 estimates the attenuation coefficient μ_{eff} based on the blood reflectance R_{blood}(ρ) (step 104). The method for calculating the attenuation coefficient μ_{eff} has been described above.

The control unit 4 provides the correct, approximation-error-free attenuation coefficient μ_{eff} by multiplying μ_{eff} estimated in the paragraph described above by the calibration coefficient (step 105).

The control unit 4 calculates the equivalent scattering coefficient μₛ′(λ₁) and absorption coefficient μₐ(λ₁) from μ_{eff} (λ₁) and μ_{eff}(λ₂) provided by carrying out the process described above using light sources that output light having the two wavelengths λ₁ and λ₂ (step 106). The methods for calculating the equivalent scattering coefficient μₛ′(λ₁) and the absorption coefficient μₐ(λ₁) have been described above.

As described above, the measuring device and method according to the present embodiment allow precise measurement of the blood turbidity.

An embodiment has been described above, and the embodiment is presented by way of example and is not intended to limit the scope of the invention. The novel embodiment can be implemented in a variety of other forms, and a variety of omissions, replacements, and changes can be made to the embodiment to the extent that they do not depart from the substance of the invention. The embodiment and variations thereof are encompassed in not only the scope and substance of the invention but the invention described in the claims and the scope equivalent thereto.

### REFERENCE SINGS LIST

1 Noninvasive measuring device
2 Light radiator
3 Optical intensity detector
4 Control unit

1. A noninvasive measuring device comprising:
a light radiator that radiates light to a predetermined position on a subject;
optical intensity detectors that are disposed in positions above a blood vessel of the subject and detect a first optical intensity of light emitted from the subject in positions separate at predetermined intervals or in continuous positions from a position irradiated with the light from the light radiator;
optical intensity detectors that are disposed in positions not above the blood vessel of the subject and detect a second optical intensity of light emitted from the subject in positions separate at predetermined intervals or in continuous positions from the position irradiated with the light from the light radiator; and
a control unit that calculates a third optical intensity from blood in the blood vessel by using the first optical intensity and the second optical intensity.

2. The noninvasive measuring device according to claim 1,
wherein the control unit calculates the third optical intensity by using Numerical Formula 1 below,
$${R}_{blood} ( ρ ) = \frac{( 1 - β ) {R}_{on} - ( 1 - α ) {R}_{off}}{α - β} \qquad [ Numerical Formula 1 ]$$
where R_{blood}(ρ) represents the third optical intensity, Rₒₙ represents the first optical intensity, R_{off} represents the second optical intensity, and α and β represent values determined in advance.

3. The noninvasive measuring device according to claim 1, wherein the control unit calculates an attenuation coefficient from the third optical intensity.

4. The noninvasive measuring device according to claim 3, wherein the control unit calibrates the attenuation coefficient by multiplying the attenuation coefficient by a calibration coefficient.

5. The noninvasive measuring device according to claim 3,
wherein the light radiator radiates light having a first wavelength and light having a second wavelength, and
the control unit
calculates a first attenuation coefficient in accordance with which the light having the first wavelength is attenuated and a second attenuation coefficient in accordance with which the light having the second wavelength is attenuated, and
separates and calculates an equivalent scattering coefficient or an absorption coefficient for the blood from the first attenuation coefficient and the second attenuation coefficient.

6. A noninvasive measuring program that causes a computer in a device including a light radiator that radiates light to a predetermined position on a subject, optical intensity detectors that are disposed in positions above a blood vessel of the subject and detect a first optical intensity of light emitted from the subject in positions separate at predetermined intervals from a position irradiated with the light from the light radiator, and optical intensity detectors that are disposed in positions not above the blood vessel of the subject and detect a second optical intensity of light emitted from the subject in positions separate at predetermined intervals from the position irradiated with the light from the light radiator to carry out the process of calculating a third optical intensity from blood in the blood vessel by using the first optical intensity and the second optical intensity.

7. A noninvasive measuring method comprising:
radiating light to a predetermined position on a subject;
detecting a first optical intensity of light emitted from the subject in positions on a blood vessel of the subject and separate at predetermined intervals from a position irradiated with the light;
detecting a second optical intensity of light emitted from the subject in positions not on the blood vessel of the subject and separate at predetermined intervals from the position irradiated with the light; and
calculating a third optical intensity from blood in the blood vessel by using the first optical intensity and the second optical intensity.

8. The noninvasive measuring device according to claim 2, wherein the control unit calculates an attenuation coefficient from the third optical intensity.

9. The noninvasive measuring device according to claim 8, wherein the control unit calibrates the attenuation coefficient by multiplying the attenuation coefficient by a calibration coefficient.

10. The noninvasive measuring device according to claim 9,
wherein the light radiator radiates light having a first wavelength and light having a second wavelength, and
the control unit
calculates a first attenuation coefficient in accordance with which the light having the first wavelength is attenuated and a second attenuation coefficient in accordance with which the light having the second wavelength is attenuated, and
separates and calculates an equivalent scattering coefficient or an absorption coefficient for the blood from the first attenuation coefficient and the second attenuation coefficient.

11. The noninvasive measuring device according to claim 8,
wherein the light radiator radiates light having a first wavelength and light having a second wavelength, and
the control unit
calculates a first attenuation coefficient in accordance with which the light having the first wavelength is attenuated and a second attenuation coefficient in accordance with which the light having the second wavelength is attenuated, and
separates and calculates an equivalent scattering coefficient or an absorption coefficient for the blood from the first attenuation coefficient and the second attenuation coefficient.

12. The noninvasive measuring device according to claim 4,
wherein the light radiator radiates light having a first wavelength and light having a second wavelength, and
the control unit
calculates a first attenuation coefficient in accordance with which the light having the first wavelength is attenuated and a second attenuation coefficient in accordance with which the light having the second wavelength is attenuated, and
separates and calculates an equivalent scattering coefficient or an absorption coefficient for the blood from the first attenuation coefficient and the second attenuation coefficient.
